(19) United States
(12) Patent Application Publication
(10) Pub. No.: US 2004/0110847 A1
(43) Pub. Date: Jun. 10, 2004

(54) Compounds extracted from Sap of Rhus succedanea

(76) Inventors: Pei-Lin Wu, Tainan (TW); Shwu-Bin Lin, Taipei (TW); Robin Yih-Yuan Chiou, Chia Yi City (TW)

Correspondence Address: THORP REED & ARMSTRONG, LLP, ONE OXFORD CENTRE, 301 GRANT STREET, 14TH FLOOR, PITTSBURGH, PA, 15219-1425, US

(21) Appl. No.: 10/166,918

(22) Filed: Jun. 11, 2002

(51) Int. Cl. A61K 31/05; C07C 39/08

(52) U.S. Cl. 514/734; 568/766

(57) ABSTRACT

The present invention relates to new antioxidative and cytotoxic hydroquinone compounds 10′(Z),13′(E),15′(E)-heptadecatrienylhydroquinone (1) and 10′(Z),13′(E)-heptadecadienylhydroquinone (2) that were mainly isolated from an ethanol extract of the sap of Rhus succedanea. The structures of these compounds were determined by spectral analyses and showed potent antioxidative activity and cytotoxicity against human cancer cells. This invention also includes anti-tumor pharmaceutical composition of the hydroquinone compounds.

### BACKGROUND OF THE INVENTION

1. Field of the Invention

This invention relates to two new compounds extracted from the Sap of Rhus succedanea, and more particularly to two new compounds having antioxidative and cytotoxic functions characteristics in pharmaceutical application.

2. Description of Related Art

The lacquer trees grown in Puli, Nantao, Taiwan are in the species Rhus succedanea that originated in Vietnam and are different from Rhus vernicifera mainly grown in China, Korea and Japan.¹⁻³ The sap collected from lacquer trees, called oriental or natural lacquer, has been used as adhesives and coatings for more than 5000 years in China and Japan. Since the Han Dynasty of China, as recorded in an ancient medical article “Sennong Bercao Jin,” the sun-dried lacquer after being ground into powder has been used as a folk medicine for various therapeutic purposes.

Urushiol, the major component in the lacquer of Rhus vernicifera, was found to be phenolic lipid that is a mixture of 3-substituted catechols containing C₁₅-carbon chains with 0 to 3 double bonds.⁴ In general, the triolefinic urushiol content was found to be 60-70% and contaminated with other phenolic lipids depending on the locations where the lacquer trees were grown.

Cancer in the form of malignant tumors transfers to other organs and tissue by the blood and lymph circulatory system to damage cells, disrupt organs' work and finally terminates the life of the infected individual. Cancer has various types that are typically divided into solid tumors and malignant blood cells. The solid tumors often found in clinics are divided into four types that follow.

(1) Cervical epithelioid carcinoma: the cervical epithelioid carcinoma is a gynecologic malignancy of the female genital tract.

(2) Hepatoma: hepatoma is the most common cancer in males and the third common cancer in females in Taiwan.

(3) Colorectal cancer: colorectal cancer is a significant cancer in Western population. It develops as the result of a pathologic transformation of normal colon epithelium to an invasive cancer.

(4) Colon adenocarcinoma: cancer of the colon is common in the western world and is an important cause of morbidity and mortality, having an incidence of about 5% in the U.S. population.

Traditional therapies for cancer include surgery, radiation therapy, chemotherapy, immunotherapy and gene therapy. Chemotherapy is often performed in conjunction with other therapies to cure patients suffering from cancer. Chemotherapy uses chemicals or medicine possessing cytotoxic properties to kill or control the cancer cells. It is know that extracts from lacquer trees contain some compounds having cytotoxic properties and these compounds are suitable for use in the clinical treatment of patients with cancer.

### SUMMARY OF THE INVENTION

The main objective of the present invention is to disclose two new hydroquinones compounds, 10′(Z),13′(E),15′(E)-heptadecatrienylhydroquinone and 10′(Z),13′(E)-heptadecadienylhydroquinone, obtained from the sap of Rhus succedanea.

Another objective of the present invention is to disclose the curative features of the two new compounds, 10′(Z),13′(E),15′(E)-heptadecatrienylhydroquinone (1) and 10′(Z),13′(E)-heptadecadienylhydroquinone (2) with regard to human cancer cell lines.

Further benefits and advantages of the present invention will become apparent after a careful reading of the detailed description.

### BRIEF DESCRIPTION OF THE FIGURES

FIG. 1 is the structure of hydroquinones 1 and 2 showing the major HMBC correlations.

FIG. 2 is a graph of the antioxidative potency (AOP) of hydroquinone compounds 1 and 2 and butylated hydroxytoluene.

### DETAILED DESCRIPTION OF THE INVENTION

Antioxidant-directed fractionation of the 80% ethanol extract from the sap of Rhus succedanea by HPLC analysis afforded two new compounds 10′(Z),13′(E),15′(E)-heptadecatrienylhydroquinone (1) and 10′(Z),13′(E)-heptadecadienylhydroquinone (2) as well as a known 10′(Z)-heptadecenylhydroquinone (3).⁵ The isolation, structural elucidation and antioxidative and cytotoxic activities of these hydroquinone compounds are described.

The experimental processes are described as follows to show the means of extracting the new compounds and the cytotoxic activity of the new compounds.

(1). General Experimental Method: The UV spectra were obtained on an Agilent 8453 spectrophotometer. The IR spectra were measured on a Jasco FT/IR-200E spectrophotometer using a thin film on a KBr disc. ¹H and ¹³C NMR, DEPT, COSY, HMQC, HMBC, and NOESY spectra were recorded on either Bruker Avance-300 or AMX-400 FT-NMR instruments. All chemical shifts were reported in ppm from tetramethylsilane as an internal standard. Low- and high-resolution MS were performed in the EI mode on either a Finnigan Trace or VG 70-250S spectrometer. High performance liquid chromatographic (HPLC) analyses were performed on an L-7100 HPLC pump in connection with an L-7420 UV/VIS detector monitored at 254 nm (Hitachi Co. Ltd., Tokyo, Japan) and a reverse phase C₁₈ column (Hypersil ODS column, 250×4.6 mm, 5 μm, Thermo Hypersil Ltd., England) or a semi-preparative column (Hypersil ODS column, 250×11.0 mm, 5 μm, Thermo Hypersil Ltd., England). Sonication was done with an Ultrasonic Cleaner (B-42, Branson Cleaning Equipment Company, Shelton, Conn., USA).

(2). Plant Material: The sap, natural lacquer, was obtained from Longnan Natural Lacquer Museum, Puli, Nantao, Taiwan. The lacquer was harvested from the Lac trees of Rhus succedanea grown in the Puli area by cutting the trunk skin of lacquer trees into strips and collecting the outflow.

(3). Extraction and Isolation: An aliquot of the lacquer (10 g) was dissolved and mixed thoroughly with 90 mL of 80% ethanol at ambient temperature. The upper layer was withdrawn and centrifuged (8000 g, 5 min). The supernatant was diluted 20 fold with 80% methanol and subjected to HPLC analysis run with a gradient solvent of 50 to 90% of aqueous methanol (v/v). The flow rate and injection volume were 1 ml/min and 20 μl, respectively. When the peak-fractions were collected and subjected to antioxidative determination by the procedure described below, two peaks with potent antioxidative activity were observed. Then, the two active fractions were separated by subjecting the supernatant to a semi-preparative HPLC under an isocratic condition (methanol/water, 80:20, v/v) with 3 ml/min of flow rate and 2.5 ml of injection volume. These fractions were further purified by running through the same column under an isocratic condition (methanol/water, 85/15, v/v) to collect the separate fractions and were lyophilized with a freeze drier. The estimated yields for compounds 1 and 2 were 22.95 and 31.45 mg/g sap, respectively. Apparently, the sap of Rhus succedanea is a potent source of the hydroquinones.

The experimental data are shown in Table 1 and Table 2 and listed as follows:

TABLE 1
¹H and ¹³C NMR Data (CD₃OD, δ, multiplicity,
J, Hz) of the Hydroquinone Compounds 1-2 Extracted
and Purified from the Sap of Rhus succedanea.

| | Compounds and analyses | | | |
|---|---|---|---|---|
| | 1 | | 2 | |
| | δ_{H} | δ_{C} | δ_{H} | δ_{C} |
| 1 | | 144.3 | | 144.3 |
| 2 | | 130.7 | | 130.7 |
| 3 | 6.56 d (2.8 Hz) | 121.9 | 6.55 d (2.4) | 121.9 |
| 4 | | 145.9 | | 145.9 |
| 5 | 6.61 dd (7.2, 2.8) | 113.6 | 6.61 dd (7.2, 2.4) | 113.6 |
| 6 | 6.58 d (7.2) | 120.1 | 6.58 d (7.2) | 120.1 |
| 1′ | 2.58 t (7.6) | 31.1 | 2.57 t (7.2) | 31.0 |
| 2′ | 1.58 quintet (7.6) | 31.1 | 1.58 quintet (7.2) | 31.1 |
| 3′-8′ | 1.30 m | 30.6-30.8 | 1.31 m | 30.3-30.7 |
| 9′ | 2.05 q (6.8) | 28.0 (27.2^{a}) | 2.04 q (6.8) | 28.0 (27.2^{a}) |
| 10′ | 5.41 m | 131.8 | 5.38 m | 131.3 |
| 11′ | 5.37 m | 128.1 | 5.38 m | 128.8 |
| 12′ | 2.79 t (6.8) | 31.2 (31.9^{a}) | 2.73 t (6.0) | 31.3 (31.9^{a}) |
| 13′ | 5.49 m | 130.7 | 5.38 m | 129.8 |
| 14′ | 5.99 m | 131.8 | 5.38 m | 131.4 |
| 15′ | 5.99 m | 132.9 | 1.96 q (6.0) | 35.8 (34.6^{a}) |
| 16′ | 5.55 m | 127.7 | 1.31 m | 23.8 |
| 17′ | 1.70 d (6.4) | 18.1 (18.0^{a}) | 0.89 t (7.6) | 14.0 |

^{a}The chemical shift measured in CDCl₃.

10′(Z),13′(E),15′(E)-heptadecatrienylhydroquinone (1): Pale yellow oil; UV (CHCl₃) λₘₐₓ (log ε) 247 (4.02), 277 (3.64), 319 (2.93) nm; IR (film) νₘₐₓ 3499, 3012, 2925, 2852, 1594, 1475, 1278, 986, 732 cm⁻¹; EIMS m/z (relative intensity) 342 M⁺ (12), 313 (3), 163 (18), 149 (19), 136 (31), 123 (100), 107 (36), 93 (52), 79 (71), 77 (39), 72 (70), 67 (54), 59 (75), 55 (64); HREIMS m/z 342.2588 (calcd for C₂₃H₃₄O₂, 342.2559). ¹H and ¹³C NMR data, see Table 1.

10′(Z),13′(E)-heptadecadienylhydroquinone (2): Colorless oil; UV (CHCl₃) λₘₐₓ (log ε) 242 (3.40), 274 (3.41) nm; IR (film) νₘₐₓ 3498, 3013, 2925, 2853, 1475, 1279, 965, 731 cm⁻¹; EIMS m/z (relative intensity) 344 M⁺ (6), 241 (8), 163 (8), 136 (15), 123 (100), 109 (11), 95 (21), 81 (23), 72 (54), 67 (38), 59 (95), 55 (50); HREIMS m/z 344.2714 (calcd for C₂₃H₃₆O₂, 342.2715). ¹H and ¹³C NMR data, see Table 1.

10′(Z),13′(E),15′(E)-Heptadecatrienylhydroquinone (1) was isolated as pale yellow oil. The HREIMS showed a molecular ion at m/z 342.2588 consistent with the molecular formula of C₂₃H₃₄O₂. A base peak in mass spectrum was observed at m/z 123 which corresponded to the dihydroxytropylium ion C₇H₅(OH)₂⁺. Examining the ¹H NMR spectrum, three aromatic protons at δ_{H} 6.56 (1H, d, J=2.8 Hz), 6.58 (1H, d, J=7.2 Hz) and 6.61 (1H, dd, J=7.2, 2.8 Hz) comprised a 1,2,4-trisubstituted benzene (Table 1).

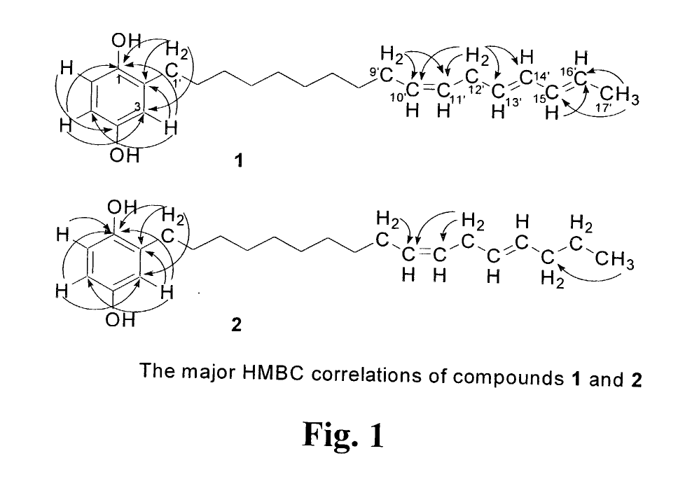

The ¹³C NMR spectrum revealed that the substituents were two phenolic hydroxyl groups (δ 144.3 and 145.9) and a carbon chain (δ 130.7). According to the molecular weight, the carbon chain containing seventeen carbons with three double bonds was suggested. A downfield-shifted doublet methyl at δ 1.70 (J=6.4 Hz, H-17′) indicated a double bond between C-15′ and C-16′. A quartet methylene at δ 2.05 (6.8 Hz, H-9′) adjacent to a double bond and a triplet methylene at δ 2.79 (J=6.8 Hz, H-12′) adjacent to two double bonds indicated that the other two double bonds located at carbons 10′ and 11′ as well as 13′ and 14′. The positions of the substituents were determined by the HMBC and NOESY experiments (FIG. 1). The distinct cross peaks between aromatic H-3 (δ 6.56) and benzylic H-1′ (δ 2.58) in the NOESY spectrum together with the ³J correlations between benzylic H-1′ and aromatic C-1 (δ 144.3), C-3 (δ 121.9) in the HMBC spectrum showed a 10′,13′,15′-heptadecatrienylhydroquinone skeleton.

The geometry of the double bonds on the linear side chain was determined as follows. The first evidence that favored 10′(Z), 13′(E) and 15′(E) configurations came from the comparison of the pattern of olefinic protons completely identical to that of 3-[8′(Z),11′(E),13′(E)-pentatrienyl] catechol.⁶ Furthermore, the upfield-shifted carbon signal of allylic C-9′ (δ 27.2 in CDCl₃) and the downfield-shifted carbon signals of C-12′ (δ 31.9 in CDCl₃) and C-17′ (δ 18.0 in CDCl₃) in compound 1 (Table 1) with respect to the analogous carbons C-9′ and C-12′ (δ 29-30), C-17′ (δ 14) in saturated heptadecylcatechol concluded the double bond configurations as 10′(Z), 13′(E) and 15′(E) based on Rossi's method.⁷ The third evidence came from decoupling experiments. An apparent doublet with J=9.6 Hz for H-10′ by irradiation of H-9′, a doublet with J=13.8 Hz for H-13′ by irradiation of H-12′ and a doublet with J=12.4 Hz for H-16′ by irradiation of H-17′ supported the stereochemistry of the double bonds between carbons 10′ and 11′; 13′ and 14′; 15′ and 16′ as Z, E, and E, respectively. Finally, the presence of NOE between H-9′ and H-12′ verified the cis configuration of the double bond between C-10′ and C-11′. The full assignments of ¹H and ¹³C NMR signals were achieved by DEPT, COSY, HMQC, HMBC, and NOESY experiments.

10′(Z), 13′(E)-Heptadecadienylhydroquinone (2) exhibited the molecular formula C₂₃H₃₆O₂ with two mass units more than compound 1 by HREIMS. Based on the analysis of the ¹H and ¹³C NMR spectra, the similarity between compounds 1 and 2 revealed that compound 2 possessed two double bonds in the C₁₇ side chain. With reference to FIG. 1, the downfield-shifted chemical shift of H-9′ (δ 2.04), H-12′ (δ 2.73) and H-15′ (δ 1.96) as well as the ³J HMBC correlation between H-17′ (δ 0.89) and C-15′ (δ 35.8) indicated two double bonds located between C-10′ and C-11′; C-13′ and C-14′. The presence of NOE between H-9′ and H-12′ referred the cis configuration of C-10′—C-11′ double bond, whereas the absence of NOE between H-12′ and H-15′ disclosed the trans configuration of C-13′—C-14′ double bond. According to Rossi's method,⁷ the upfield-shifted carbon signal of allylic C-9′ (δ 27.2 in CDCl₃) and the downfield-shifted carbon signals of C-15′ (δ 34.6 in CDCl₃) in compound 1 (Table 1) with respect to the analogous carbons C-9′ (δ 29-30) and C-15′ (δ 32) in saturated heptadecylcatechol further supported the 10′(Z) and 13′(E) stereochemistry.

(4). Determination of Antioxidative Activity: The procedure using an iron/ascorbate system reported by Yoon,⁹ Chiou,¹⁰ Decker,¹¹ Tamura,¹² and Sasaki¹³ was modified. In a beaker (50 ml), 100 mg of linoleic acid, 1.0 g of Tween 20, and 20 ml of Tris-buffer (pH 7.4, 50 mM) were combined, gently shaken and emulsified with a sonicator for 2 min. An iron/ascorbate solution containing 30 μM FeCl₃ and 200 μM ascorbic acid in the Tris-buffer (pH 7.4, 50 mM) was prepared daily. In a series of 1.5-ml microfuge tubes, 0.5 ml of the emulsified linoleic acid, 0.5 ml of the iron/ascorbate solution, and 0.1 ml of methanol containing various concentrations of the three compounds or BHT including 0, 10, 40 and 100 ppm (μg/ml) (resulting in ca. 0, 1, 4 and 10 ppm of concentration after combination) were deposited. Prior to incubation, the tubes were immersed and kept in an ice bath to minimize any reactions. To initiate reaction, the mixtures in the tubes were mixed by hand (gently to prevent foaming) and placed into the wells of a thermal block at 37° C. for 5 and 30 min. After incubation, the produced conjugated diene hydroperoxide (CDHP) contents were determined spectrophotometrically. A 0.1 ml sample of the resulting solution was withdrawn and mixed with 2.4 ml methanol. The absorbance (A) at 234 nm of the mixture was measured. Aliquots (0.1 ml) of methanol without antioxidant and methanol containing 2000 ppm of BHT were introduced and incubated concurrently as blank and control samples. All test data were the average of triplicate analyses. The percentage of AOP (capacity to inhibit peroxide formation in linoleic acid) was calculated as follows.

AOP(%)=(1−A_{234 nm} with antioxidant/A_{234 nm} without antioxidant)×100.

(5). Determination of Cytotoxicity. Four human cancer cell lines, HeLa, Huh7, HCT116 and LoVo were used to assess cytotoxicity of the purified compounds. All the cell lines, except Huh7 cells,¹⁴ were purchased from American Type Culture Collection. The cells were cultured in Dulbecco's modified Eagle medium (DMEM) supplemented with 10% fetal calf serum (FCS), 100 units/ml penicillin and 100 mg/ml streptomycin at 37° C. under 5% CO₂. Cells were grown in a 96 well plate (2000 cells/well) with or without the addition of compounds 1, 2 or 3 for 72 h. An acid phosphatase assay^{14,15} was then used to quantitate the viable cells. Briefly, after the media were aspirated, the cells were washed with phosphate buffer saline (PBS) and then incubated with 100 μl of 0.1M sodium acetate buffer containing 10 mM p-nitrophenyl phosphate and 0.1% Triton X-100, and incubated at 37° C. for 1 h. The reaction was stopped by the addition of 10 μl of 1 N NaOH and coloration of the cells was measured at 410 nm by an ELISA reader. The concentrations of 50% growth inhibition (IC₅₀) were interpolated from dose-dependent growth inhibition curves. The data HeLa, Huh7, HCT116 and LoVo presented were the averages derived from a minimum of triplicate independent experiments.

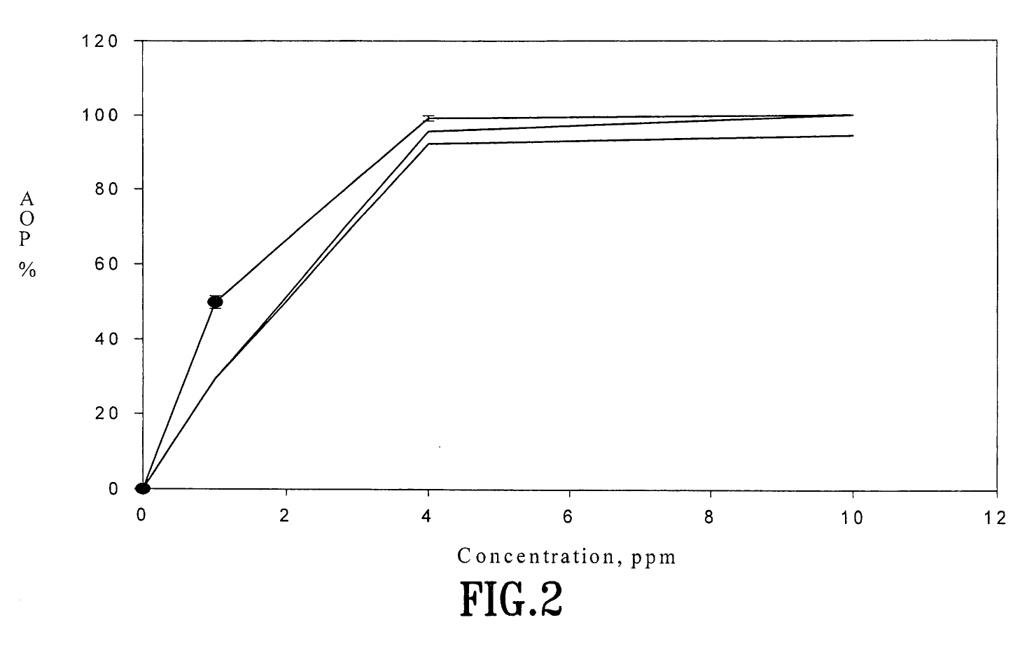

The two hydroquinone compounds 1-2 and a known antioxidant, butylated hydroxytoluene (BHT), were subjected to an iron/ascorbate system using linoleic acid as substrate for antioxidative potency (AOP) determination. With reference to FIG. 2, the AOPs of the hydroquinones 1-2 were close to that of BHT. This indicated that the two hydroquinones were potent antioxidants.

(6). Conclusion:

Hydroquinones 1-2 were both subjected to cytotoxicity evaluation. These compounds exhibited significant cytotoxic activity against four human cancer cell lines (or tumor) having cells which increase rapidly in an uncontrolled way and produce abnormal growth. These human cancer cell lines specially include cervical epithelioid carcinoma (HeLa), hepatoma cell line (Huh7), colorectal cancer cell line (HCT116) and colon adenocarcinoma (LoVo). The concentrations of IC₅₀ against the cancer cell lines were 2.0 to 4.5 μg/ml, 3.5 to 6.0 μg/ml and 2.9 to 6.4 μg/ml, respectively, depending on the nature of the cells (Table 2).

TABLE 2
Cytotoxicity of the Hydroquinone Compounds
1-2 from the Sap of Rhus saccedanea
toward Four Human Cancer Lines^{a} (IC₅₀ in μg/ml)

| | Cell line^{a} and IC₅₀ in μg/ml | | | |
|---|---|---|---|---|
| Compound | Hela cells | Huh7 cells | HCT116 cells | Lovo cells |
| 1 | 2.8 | 3.9 | 2.0 | 4.5 |
| 2 | 4.6 | 6.0 | 3.5 | 5.6 |

^{a}HeLa = Human cervical epithelioid carcinoma; Huh7 = Human hepatoma cell line; HCT116 = Human colorectal cancer cell line; LoVo = Human colon adenocarcinoma

According to the foregoing description, the hydroquinone compounds 1 and 2 have excellent antioxidative and cytotoxic efficiency, especially cytotoxic efficiency against human cancer lines. Thus, the hydroquinone compounds can be selectively prepared into an anti-tumor composition to act as an anti-tumor agent or combined into acceptable esters and salt of the hydroquinone compounds 1 and 2. These hydroquinone compounds 1 and 2, esters, and salts thereof are selectively combined with other pharmaceutically acceptable salts esters and carriers in the anti-tumor composition in treating people suffering cancer such as cervical epithelioid carcinoma (HeLa), hepatoma (Huh7), colorectal cancer (HCT116) and colon adenocarcinoma (LoVo). Examples of such salts include inorganic acid salts such as hydrochloride, sulfate and nitrate as well as organic acid salts such as maleate and tartrate. Other salts also may be used insofar as they are pharmaceutically acceptable. The anti-tumor pharmaceutical composition comprises an anti-tumor effective amount of at least one of the hydroquinone compounds as described above, or a pharmaceutically acceptable salt or ester thereof.

The pharmaceutical composition of the present invention can be administered either orally or parenterally. As for their dosage forms, they may be prepared in any of such forms as solid forms like tablets, granules, powder and capsules or liquid forms like injection, by conventional methods. Such preparation may contain usual additives such as excipients, binders, thickeners, dispersing agents, resorption enhancers, buffering agents, surfactants, solubilizers, preservatives, emulsifiers, isotonizers, stabilizers and pH adjusting agents. The amount of compounds 1 and 2 are administered depending on the concentrations of IC₅₀ required to effectively combat the cancer cell lines for cervical epithelioid carcinoma (HeLa), hepatoma (Huh7), colorectal cancer (HCT116) and colon adenocarcinoma (LoVo) in clinic therapy.

Various modifications and variations of the present invention will be recognized by those persons skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

### References and Notes

(1) Hsu, Y. -F. Taiwan Handicraft Ind. 1994, 54, 26-32.

(2) Kumanotani, J. Prog. Org. Coatings 1998, 34, 135-146.

(3) Oshima, R.; Yamauchi, Y.; Watanabe, C.; Kumanotani, J. J. Org. Chem. 1985, 50, 2613-2621.

(4) Yamauchi, Y.; Oshima, R.; Kumanotani, J. J. Chromatogr. 1982, 243, 71-84.

(5) David, J. M.; Chavez, J. P.; Chai, H. -B.; Pezzuto, J. M.; Cordell, G. A. J. Nat. Prod 1998, 61, 287-289.

(6) Du, Y.; Oshima, R.; Kumanotani, J. J. Chromatogr 1984, 284, 463-473.

(7) Rossi, R.; Carpita, A.; Quirici, M. G.; Veracini, C. A. Tetrahedron 1982, 38, 639-644.

(8) Buser, H. R.; Arn, H.; Guerin, P.; Rauscher, S. Anal. Chem. 1983, 55, 818-822.

(9) Yoon, S. H.; Kim, S. K.; Shin, M. G.; Kim, K. H. J. Am. Oil Chem. Soc. 1985, 62, 1487-1489.

(10) Chiou, R. Y. -Y.; Shyu, S. L.; Tsai, C. L. J. Food Sci. 1991, 56, 1375-1377.

(11) Decker, E. A.; Faraji, H. J. Am. Oil Chem. Soc. 1991, 67, 650-652.

(12) Tamura, H.; Kitta, K.; Shibamota, T. J. Agric. Food Chem. 1991, 39, 439-442.

(13) Sasaki, S.; Ohta, T.; Decker, E. A. J. Agric. Food Chem. 1996, 44, 1682-1686.

(14) Lin, S. B.; Hsieh, S. H.; Hsu, H. L.; Lai, M. Y.; Kan, L. S.; Au, L. C. J. Biochem. 1997, 122, 712-722.

(15) Connolly, D. T.; Knight, M. B..; Harakas, N. K.; Wittwer, A. J.; Feder, J. Anal. Biochem. 1986, 152, 136-140.

What is claimed is

1. A hydroquinone compound substantially having the general formula of: 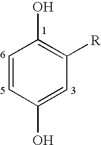
wherein r is 10′(Z),13′(E),15′ (E)-heptadecatrienyl or 10′(Z),13′(E)-heptadecadienyl, and a pharmaceutically acceptable salt or ester thereof.

2. The hydroquinone compound as claimed in claim 1, which is 10′(Z), 13′(E), 15′(E)-heptadecatrienylhydroquinone.

3. The hydroquinone compound as claimed in claim 1, which is 10′(Z), 13′(E)-heptadecadienylhydroquinone.

4. The hydroquinone compound as claimed in claim 2, which is isolated from the sap of Rhus succedanea.

5. The hydroquinone compound as claimed in claim 3, which is isolated from the sap of Rhus succedanea.

6. The hydroquinone compounds as claimed in claim 1 for use in the preparation of a pharmaceutical composition for the treatment of a patient having a tumor.

7. An anti-tumor pharmaceutical composition, which comprises an anti-tumor effective amount of at least one of the hydroquinone compounds as claimed in claim 1, or a pharmaceutically acceptable salt or ester thereof.

8. The anti-tumor pharmaceutical composition as claimed in claim 7 for use in the treatment of a patient having cervical epithelioid carcinoma, hepatoma, colorectal cancer or colon adenocarcinoma.

9. The anti-tumor pharmaceutical composition as claimed in claim 7 further containing other known anti-tumor pharmaceutical compositions.

10. A method of isolating the hydroquinone compounds in claim 1 comprising steps:
collecting sap of Rhus succedanea;
dissolving the sap in ethanol solution to become a mixture, then withdrawing an upper layer solution of the mixture to centrifuge;
collecting supernatant from the upper layer solution after centrifuge; and
analyzing the supernatant with HPLC analysis run and purifying hydroquinone compound from the supernatant.

11. The method as claimed in claim 10, wherein the HPLC analysis run having a gradient solvent of 50 to 90% of aqueous methanol (v/v) and the flow rate and injection volume were 1 ml/min and 20 μl, respectively; and
subjecting the supernatant to a semi-preparative HPLC with 3 ml/min of flow rate and 2.5 ml of injection volume.
